(19) United States
(12) Patent Application Publication
(10) Pub. No.: US 2022/0399130 A1
(43) Pub. Date: Dec. 15, 2022

(54) EXPERT BOARD CASE SELECTION SYSTEM AND METHOD

(71) Applicant: ULTIMATE OPINIONS IN MEDICINE LLC, Boca Raton, FL (US)

(72) Inventors: Mohammad Jahanzeb, Boca Raton, FL (US); Afzaal Akhtar, Boca Raton, FL (US)

(21) Appl. No.: 17/840,481

(22) Filed: Jun. 14, 2022

Related U.S. Application Data
(60) Provisional application No. 63/210,838, filed on Jun. 15, 2021.

(51) Int. Cl. G16H 50/70; G06F 16/33

(52) U.S. Cl. CPC G16H 50/70; G06F 16/3347

(57) ABSTRACT

The present embodiments relate to generation of a docket of feature groups based on a plurality of identified features from a dataset. For instance, the docket can be used by a board of experts to efficiently review and provide insights to a larger number of cases. Data relating to each of the plurality of scenarios can be processed to extract textual features for each of the plurality of scenarios. Feature vectors can be populated with textual features from the plurality of scenarios, and feature groups can be derived by a docket generation model. The docket generation model can generate a docket comprising a listing of the set of feature groups. The listing of the set of feature groups can be arranged by a number of scenarios corresponding to each of the feature groups and/or a number of extracted textual features for each of the set of feature groups.

### CROSS-REFERENCE TO RELATED APPLICATION(S)

The present application claims priority to U.S. Provisional Patent No. 63/210,838, titled “EXPERT BOARD CASE SELECTION SYSTEM AND METHOD” and filed Jun. 15, 2021, the entirety of which is incorporated by reference hereto.

### FIELD

The disclosure generally relates to determining topics for an expert board. More particularly, the present disclosure relates to processing datasets to automatically generate dockets comprising a set of topics derived from the datasets.

### BACKGROUND

A computer or network of computing devices can store and maintain large volumes of data. The dataset can further include various types of data, such as structured and unstructured datasets, tables, etc. For example, a dataset can provide medical data for a series of subjects (e.g., cancer patients).

The dataset can include various features that are common across groups of the subjects (e.g., features common to a group with a common type of cancer, features common to a group with a common co-morbidity). As another example, the dataset can include data relating to a series of automatically generated tickets for a datacenter. In this example, features can be common across groups of the automatically generated tickets (e.g., features for a group of tickets identifying a specific server or network component, features for a group of tickets relating to a service type). However, in many instances, deriving insights into large datasets can be resource intensive.

### SUMMARY

The present embodiments relate to generation of a docket of feature groups based on a plurality of identified features from a dataset. In a first example embodiment, a method is provided.

The method can include receiving data relating to a plurality of scenarios. In some instances, the scenarios relate to medical cases, and the supporting files comprise medical documents. In other instances, the scenarios comprise automatically-generated tickets relating to a datacenter, and the supporting files comprise data relating to the automatically-generated ticket.

The method can also include processing the data relating to each of the plurality of scenarios to extract a set of textual features for each of the plurality of scenarios. Each extracted textual feature can include a feature specific to a scenario.

In some instances, processing the data relating to each of the plurality of scenarios to extract the set of textual features for each of the plurality of scenarios further comprises converting each supporting file into unstructured text. The processing can be performed on the unstructured text of each supporting file. Further, processing the data relating to each of the scenarios on the unstructured text of each supporting file further can include using a text analytics process to programmatically extract each textual feature from the unstructured text of each supporting file.

The method can also include initializing, for each of the plurality of scenarios, a feature vector comprising a plurality of null values.

The method can also include populating values of each feature vector with corresponding textual features extracted from the data relating to each of the plurality of scenarios. In some instances, a feature vector can include both null values and populated values.

In some instances, each textual feature is populated in each corresponding feature vector according to a location of the textual feature identified from each corresponding supporting file.

The method can also include processing each of the feature vectors to identify a set of feature groups. Each feature group can include a set of scenarios that include one or more extracted textual features that are common across feature vectors corresponding to each scenario of the set of scenarios.

The method can also include generating a docket comprising a listing of the set of feature groups. The listing of the set of feature groups can be arranged by a number of scenarios corresponding to each of the feature groups and/or a number of extracted textual features for each of the set of feature groups.

In some instances, processing each textual feature part of each of the feature groups can use a feature hierarchy to generate a cumulative feature score of each feature group. A feature hierarchy can include a multi-level hierarchy of features, with each feature assigned weights and/or values for use in identifying a priority of each feature group. In some instances, the feature hierarchy can utilize a tree structure, and the arrangement of feature groups on the docket can be based on a location of each feature group in the tree structure. The arrangement of the feature groups can be based at least on the cumulative feature score of each feature group. In some instances, the docket generation model includes a random forest model. In some instances, the listing of feature groups are presented as sentences that include the features common to the feature groups.

The method can also include transmitting the docket to a plurality of client devices. In some instances, the method can include obtaining a response for each of the set of feature groups listed in the docket from any of the client devices.

In some instances, the method can include training the docket generation model using the feature vectors, the set of feature groups, and the responses for each of the set of feature groups to generate a feature hierarchy of the extracted textual features in the feature vectors.

In another example embodiment, a system is provided. The system can include a processor and a computer-readable medium. The computer-readable medium can comprise instructions that, when executed by the processor, cause the processor to receive data relating to a plurality of scenarios. Each of the scenarios can comprise one or more corresponding supporting files.

The instructions can further cause the processor to process the data relating to each of the plurality of scenarios to extract a set of textual features for each of the plurality of scenarios. Processing the data further can include converting each supporting file into unstructured text.

The instructions can further cause the processor to populate, for each of the plurality of scenarios, values of a corresponding feature vector with corresponding textual features extracted from the data relating to each of the plurality of scenarios. Each textual feature can be populated in each corresponding feature vector according to a location of the textual feature identified from each corresponding supporting file.

The instructions can further cause the processor to process each of the feature vectors to identify a set of feature groups, each feature group comprising a set of scenarios that include one or more extracted textual features that are common across feature vectors corresponding to each scenario of the set of scenarios. The instructions can further cause the processor to generate a docket comprising a listing of the set of feature groups. The listing can be arranged by a number of scenarios corresponding to each of the feature groups and/or a number of extracted textual features for each of the set of feature groups.

The instructions can further cause the processor to transmit the docket to a plurality of client devices.

In some instances, processing the data relating to each of the scenarios on the unstructured text of each supporting file further comprises using a text analytics process to programmatically extract each textual feature from the unstructured text of each supporting file.

In some instances, the instructions further cause the processor to process each textual feature part of each of the feature groups using a feature hierarchy to generate a cumulative feature score of each feature group, wherein the arrangement of the feature groups is based at least on the cumulative feature score of each feature group.

In some instances, the instructions further cause the processor to obtain a response for each of the set of feature groups listed in the docket from any of the client devices.

In some instances, the instructions further cause the processor to train a docket generation model using the feature vectors, the set of feature groups, and the responses for each of the set of feature groups to generate a feature hierarchy of the extracted textual features in the feature vectors.

In another example embodiment, a computer-implemented method is provided. The computer-implemented method can include receiving at data relating to a plurality of scenarios. Each of the scenarios can comprise one or more corresponding supporting files.

The computer-implemented method can also include processing the data relating to each of the plurality of scenarios to extract a set of textual features for each of the plurality of scenarios.

The computer-implemented method can also include populating, for each of the plurality of scenarios, values of a corresponding feature vector with corresponding textual features extracted from the data relating to each of the plurality of scenarios.

The computer-implemented method can also include processing, by a docket generation model, each of the feature vectors to identify a set of feature groups, each feature group comprising a set of scenarios that include one or more extracted textual features that are common across feature vectors corresponding to each scenario of the set of scenarios, and generate a docket comprising a listing of the set of feature groups. The listing can be arranged by a number of scenarios corresponding to each of the feature groups and/or a number of extracted textual features for each of the set of feature groups.

The computer-implemented method can also include transmitting the docket to a plurality of client devices.

The computer-implemented method can also include obtaining a response for each of the set of feature groups listed in the docket from any of the client devices. The computer-implemented method can also include training the docket generation model using the feature vectors, the set of feature groups, and the responses for each of the set of feature groups to generate a feature hierarchy of the extracted textual features in the feature vectors. The feature hierarchy can be used in arranging the listing of the set of feature groups.

In some instances, processing the data relating to each of the plurality of scenarios to extract the set of textual features for each of the plurality of scenarios further comprises converting each supporting file into unstructured text. The processing can be performed on the unstructured text of each supporting file.

In some instances, processing the data relating to each of the scenarios on the unstructured text of each supporting file further comprises using a text analytics process to programmatically extract each textual feature from the unstructured text of each supporting file.

In some instances, each textual feature is populated in each corresponding feature vector according to a location of the textual feature identified from each corresponding supporting file.

### BRIEF DESCRIPTION OF THE DRAWINGS

FIG. 1 shows an embodiment of the expert group scenario selection system according to some embodiments.

FIG. 2 illustrates a method for expert group scenario selection according to some embodiments.

FIG. 3 illustrates more details of the expert group scenario selection system according to some embodiments.

FIGS. 4A and 4B illustrate examples of the form used for submitting the scenario according to some embodiments.

FIGS. 5A-5C illustrate an example of the extracting process for a feature of interest in a scenario according to some embodiments.

FIG. 6 illustrates an example of the docket/agenda for a particular expert board according to some embodiments.

FIG. 7 illustrates further details of the scenario submission process according to some embodiments.

FIG. 8 illustrates further details of the scenario docket generation and expert panel discussion processes according to some embodiments.

FIG. 9 is pseudocode of a method for generation of the topics for the scenario docket/agenda according to some embodiments.

FIG. 10 illustrates an example of the method when applied to a particular medical oncology scenario according to some embodiments.

FIG. 11 illustrates an example of a bucketing model that is collectively exhaustive partition of the possibility space for an exemplary area of expertise according to some embodiments.

FIG. 12 illustrates an example method for generation of a docket according to some embodiments.

### DETAILED DESCRIPTION OF ONE OR MORE EMBODIMENTS

A computing device or a connected computer network can obtain and maintain large datasets. For example, the dataset can provide medical data relating to a series of subjects, such as cancer patients. The data included in the datasets can include various types of data, which can be processed to derive insights into the dataset. For example, in a dataset providing medical records for cancer patients, the dataset can be processed to identify features common to groups of patients for further processing.

In many cases, a group of experts can be organized to review and discuss various topics. In one illustrative example, a group of medical doctors in a medical specialty can discuss cases of a specific type (e.g., a type of cancer) and potential treatment options for such cases. Such discussions across the group of experts can be referred to as an “expert board” or a “tumor board.” Further, in this example, a problem for these groups of experts is to find a time-efficient way for the experts to discuss and consult on a large number of related scenarios quickly and thoroughly.

As another example, dataset can relate to tickets specifying various aspects for a datacenter. For example, in responsive to an inability to access a component/application in the datacenter or if network metrics (e.g., data throughput) exceed corresponding thresholds, a ticket can be generated. A ticket can specify various metadata, such as a timestamp, a specific application, module, computer, VM, etc., related to the ticket, and a nature of the ticket (e.g., network-related issue, down server, unable to access an application). In a large datacenter or a cloud computing network, a large volume of tickets can be generated in a specific time duration. Tickets can be generated by computing systems monitoring network metrics or by users interacting with the datacenter. Insights derived from the volume of tickets can include defective applications, code, nodes/devices, etc. Further, the insights can provide details relating to attempted cyber-attacks, such as malware embedded in the datacenter or a distributed denial of service (DDOS) attack at one or more nodes in the datacenter.

In such examples, a group of experts can be convened to specify actions with respect to various aspects of the datasets. For example, a group of experts can interact on client devices over a network (e.g., the Internet) and discuss groups of scenarios included in the dataset (e.g., relating to cancer patients, relating to tickets generated in a datacenter). Further, the experts can retrieve data (e.g., documents, tables) part of the dataset from a computing node to derive further insights into aspects of the dataset.

However, in many instances, processing the volume of data in the datasets can be resource-intensive and time-intensive. In such meetings, experts may have to manually process data in the datasets to discuss various topics. For example, experts may have to review numerous documents, records, etc., for multiple scenarios to gather information for a topic. As another example, experts may manually review each generated ticket for a datacenter (e.g., chronologically) and attempt to identify aspects of the tickets. This process can comprise an inefficient use of computing/computing network resources (e.g., by retrieving multiple pieces of data from the dataset). Further, such processes can miss identifying critical features in the dataset. For example, reviewing a small portion of tickets in a datacenter may not identify an attempted cyber attack (e.g., DDOS attack) to a datacenter.

The present embodiments relate to processing datasets to automatically generate dockets comprising a set of topics derived from the datasets. For example, the dataset comprising data relating to a plurality of scenarios can be processed by a computer system to derive textual features for the plurality of scenarios. The textual features can represent various aspects relating to each scenario, such as conditions, values, etc., identified from medical records relating to a patient. The set of textual features can be populated into a corresponding feature vector. Textual feature can be added in the feature vector according to a type of data for each textual feature.

A docket generation model can further process the feature vectors to derive a docket comprising identified feature groups. For instance, a set of feature vectors can be processed to identify a set of feature groups. Each feature group can include a set of scenarios that include one or more extracted textual features that are common across feature vectors corresponding to each scenario of the set of scenarios. For example, a feature group can include a group of scenarios with a common condition (e.g., a medical condition), a common value, a common identified computing node in a datacenter, etc. The docket generation model can further generate a docket comprising a listing of the set of feature groups. The listing of the set of feature groups can be arranged by a number of scenarios corresponding to each of the feature groups and/or a number of extracted textual features for each of the set of feature groups. For instance, the docket generation model can generate a hierarchical structure of feature groups structured based on various aspects of the feature groups, such as a number of scenarios in a feature group. The generated docket can include a listing of topics based on the feature groups. The docket can be transmitted to a plurality of client devices (e.g., devices associated with a plurality of experts). In response, various actions or responses to the topics in the docket can be obtained. Further, in some instances, the docket generation model can be trained using the responses/actions for the corresponding docket.

In some embodiments, an example of the generated docket can relate to determining topics of discussion of tumor cases by a medical tumor board. It will be appreciated, however, that the present embodiments are not limited to such examples. For instance, the present embodiments can be used to determine cases/scenarios/topics for discussion by any expert panel or group in any area of expertise. Furthermore, the present embodiments may be implemented using other technologies than those disclosed below and those changes are within the scope of the disclosure.

In some instances, the present embodiments can implement a bucketing schema that can be mutually exclusive and collectively exhaustive in such a way that 1) each submitted case can be classified into exactly one bucket and 2) each of the cases in the same bucket share enough similarities that they can be discussed in a scalable, parallel way. For example, in the oncology tumor board use case, suppose 35 independent tumors were submitted for review. Without any a prior knowledge of what is similar and different about the tumors and their treatment protocols, each one may need to be discussed fully and at length in order to completely characterize the recommended medical response for each tumor. However, if there were some way to know beforehand which tumor classes have very similar treatment regimens that deviate only slightly based on individual tumor characteristics, then the tumors could be grouped together and discussed much more efficiently. This is exactly what the bucketing subprocess in the intake process can accomplish, as discussed in more detail below. The intake process can also provide the ability to upload additional unstructured files that allows for the intake form to be as short as possible. Thus, rather than requiring users to fill out pages of tedious paperwork every time they submit a case for review, the system can request a minimal set of information needed to characterize the case for discussion/review. Then, the system can allow for the convenient submission of raw additional files that it can automatically ingest and extract additional key contextual details from those files. As the ability to integrate and ingest unstructured data improves over time, the intake form will get smaller and smaller.

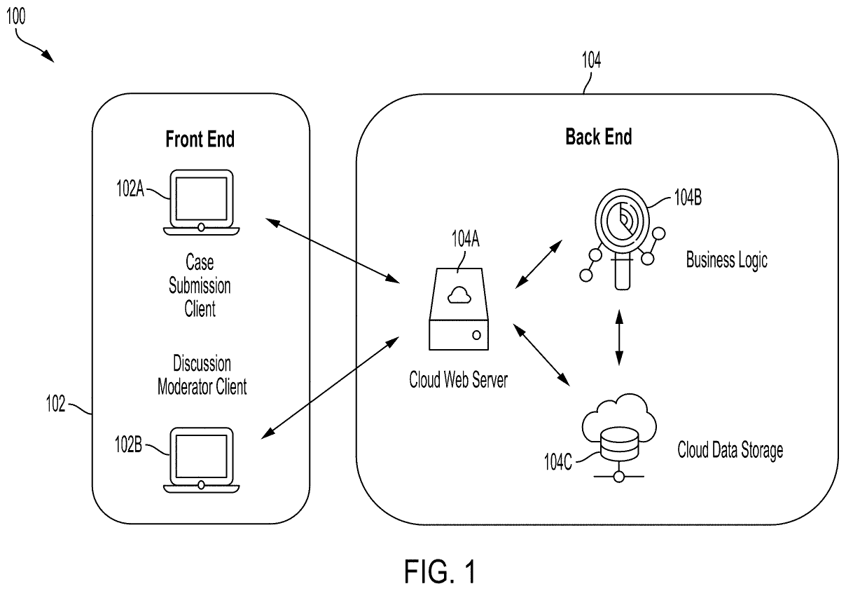

FIG. 1 shows an embodiment of the expert group scenario selection system 100. For purposes of illustration in the disclosure below, a use case of the system in which tumor cases in a medical setting that are selected for a tumor board is described. The system 100 may have a front end 102 and a backend 104 that is coupled to each other. The front end and back end 102, 104 may be located together (implemented on the same computer system or located in the same data center or company location) or may be geographically remote from each other (with the front end 102 and back end 104 each implemented on computer systems that are connected to each other (wirelessly or wired) through a communication path). Regardless of the architecture between the front end 102 and back end 104, the two connect to and communicate data and commands with each other in order to perform the processes of the system discussed below. The front end 102 may gather data/files about scenarios to be submitted to the system and allow each expert to review and analyze each selected scenario. The back end 104 may receive each submitted scenario, process and store that scenario and select the groupings of the scenarios to be analyzed by one or more experts who are part of an expert panel.

The front end 102 may further comprise a case submission client 102A, a discussion moderation client 102B and an expert client of each expert. Each of the case submission client 102A, the discussion moderation client 102B, and the expert client may be a computing device with a processor, memory, display and connectivity circuits that allow each client to couple to and communicate with the back end 104. For example, each computing device may be a smartphone, a laptop computer, a terminal, a tablet device, etc. that stores a plurality of lines of instructions that are executed by the processor that can cause the processor to be configured to perform the operations of each different client. For example, the case submission client 102A may allow each user of the system who has a scenario to submit to the expert panel to submit that scenario and any other files relevant to the submitted scenario. The user that submits a scenario may be an individual user, a representation of the individual user, a provider who is working with the individual user or providing advice to the individual user. The case submission client 102A may also present/display the results of the expert panel analysis of the scenario. Unlike other systems that may host a live audio and/or video calls about the results of an expert board's second opinion, the system may generate a video after the expert board meets, tag the video with metadata, transcribe the video, and generate summary reports, and the video and summary reports may be sent back to each case submission client 102A.

The discussion moderation client 102B may be used by an employee of the system (or a consultant) to manage the discussion process of one or more scenarios by the expert panel. Each expert client allows each expert in the expert panel to review each selected scenario and then provide their analysis for each scenario wherein the analysis varies depending on the type of scenario being submitted. For example, if each scenario is a patient cancer case, then the resultant analysis may be a recommended oncological treatment plan for that particular patient. As another example, if the scenario is an architectural plan for a new house for a user, the analysis may be one or more recommendations about that architectural plan. In all cases, the system provides more people with access to the expert panel and thus allows each user to benefit from the recommendations or second opinions of each of the experts on the expert panel.

In one embodiment, the backend 104 may further include a cloud web server 104A, business logic 104B, and cloud data storage 104C that are all implemented using cloud computing resources. These elements of the backend 104 implement processes to receive each scenario submission and one or more files, process each scenario submission and file, select the scenarios to be discussed by the expert panel, and manage the expert panel discussion as discussed below.

In one implementation of the system, each end user accesses the system's content and services via an Internet web application (an implementation of the client 102A) via mobile, tablet, or PC end points. This application hosts video, text, other multimedia content, as well as forms for digital case submission. The client 102A allows each user to submit cases for review through an online submission form that may consist of two parts: a structured intake form and unstructured additional file uploads. Each user submitting cases will fill out an intake form created by our team of field experts to quickly extract all of the key relevant details needed to assess and characterize the case. In addition, providers may upload as many additional files as they would like (.pdf, .docx, .jpeg, etc.) in any unstructured format. The information on the intake form can: 1) uniquely characterize the case into one of several “buckets” (designed through expert review for each type of domain (medical, architecture, etc.) to be a mutually exclusive and collectively exhaustive partition of the possibility space in the domain), 2) create a case record for storage in the cloud database, and 3) properly tag the case record with the bucket designation. In addition, various OCR, text analytics, and computer vision algorithms can try to pull various additional contextual pieces of information from the other submitted unstructured files to append to the case record in the database in the appropriate tables.

The system may have a number of experts who participate in the expert boards. Each expert may have a particular expertise and is chosen by the system to participate in certain expert boards based on the particular expertise of the expert and the topics being discussed by the expert board. In one embodiment, the system may tag each expert to one or more buckets in the area of expertise based on the expertise of the expert. Thus, the panel of experts may be selected by the system using the tags when a particular bucket in an area of expertise (such as HER2 breast cancer in a medical oncology expertise area) is being discussed by the expert panel.

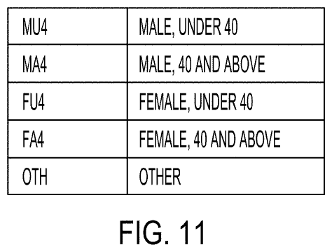

The bucketing for each area of expertise (placing each scenario into a bucket) can create the collectively exhaustive partition of the possibility space for a particular area of expertise. An example of the bucketing model is shown in FIG. 11 in which all of the possible scenarios for the area of expertise have a bucket. In the simple example in FIG. 11, all of the possible scenarios (men under 40 years old or men 40 years and above, women under 40 or women 40 years and above and other) have a bucket into which the scenarios may be placed resulting in a bucketing model that is collectively exhaustive. In a breast cancer medical oncology area of expertise example, all possible tumors may be assigned to each of a plurality of buckets so that, any breast tumor can be placed into one and only one bucket based on its characteristics, thus having a bucketing model that is collectively exhaustive for the possibility space. In each different area of expertise, there may be a similar bucketing model that is collectively exhaustive for the possibility space for that area of expertise. The bucketing model (and how many buckets exist for each bucketing model) for each area of expertise depends on the subject matter of the area of expertise. FIG. 11 shows a very simple example (5 buckets) while the breast cancer oncology area is significantly more complex (having more than 25 buckets). For each area of expertise, the bucketing model is formed for a domain expertise (cancer is one example) and a subdomain (breast cancer is one example), and the bucking model is a schema for that subdomain (types of breast cancer).

After submission of the scenarios and bucketing of the scenarios, a discussion/analysis of the scenarios for a specific bucket may be scheduled for the panel of experts. The moderator will visit the web application (the discussion moderator client 102B) using their own administrative account with privileged access and perform a “docket generation” process that can pull all submitted scenarios from the selected bucket that have not yet been processed/discussed previously and organize them into a suggested list of questions/discussion points/expected discussion times for the moderator to use when leading the round table discussion. As a non-limiting example, the selected bucket for a medical tumor board may be patients who have breast cancer and the scenarios in the bucket are discussed by experts in the field of breast cancer who provide recommended oncological treatments for each patient scenario.

A roundtable discussion may involve the discussion of several buckets, so the full docket creation process may involve segmenting the conversation into buckets. Next, within each bucket, the process may suggest a high-level introductory question to begin the discussion that will apply to all cases within that bucket. For example, if the discussion is about breast cancer tumors and the bucket is HER2+ cancer, then the initial question may be something like “What is the state-of-the-art in the treatment of HER2+ breast tumors?”

The above process may then search through the unique additional contextual facts of the cases being discussed within that bucket to identify commonalities and distinction points to generate additional follow-up questions. For the previous example, the docket may then suggest “How does your recommendation change if a patient is diabetic?” or “What if the tumor does not respond to X?”. By identifying these commonalities, the moderator can effectively allow the experts at the table to discuss multiple cases simultaneously, significantly decreasing the amount of time needed to review all submitted cases. However, the selection process is designed such that it will guarantee that all of the relevant details from any given specific case in the bucket will be discussed at some point so that each patient's scenario is fully discussed by the expert panel.

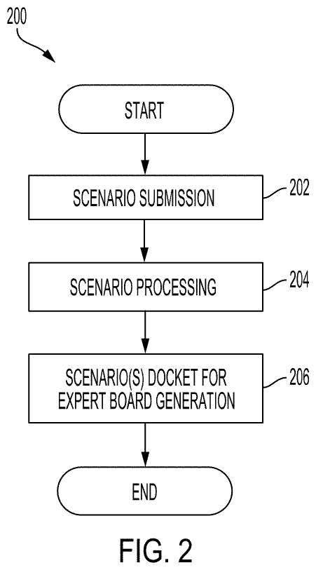

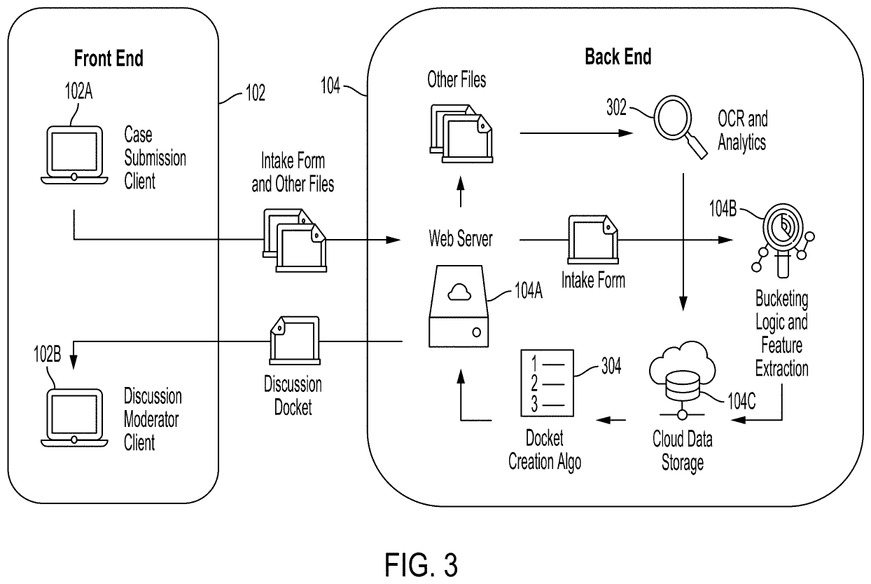

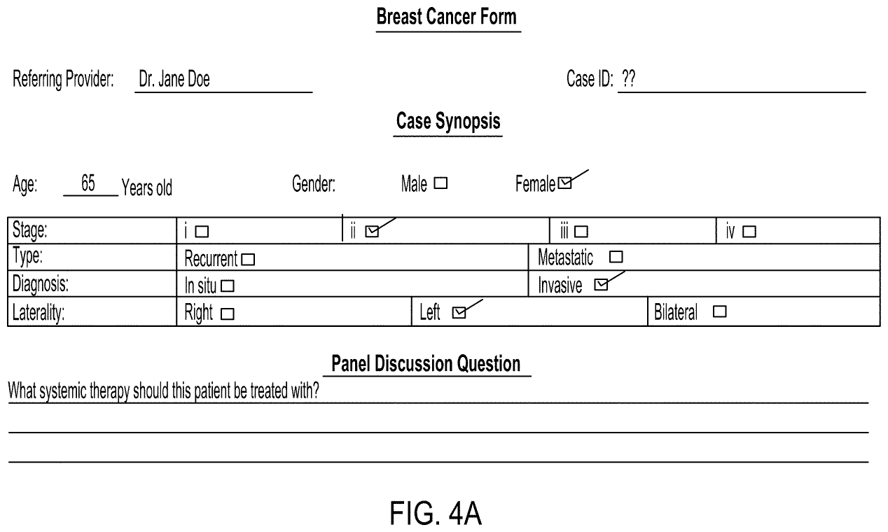

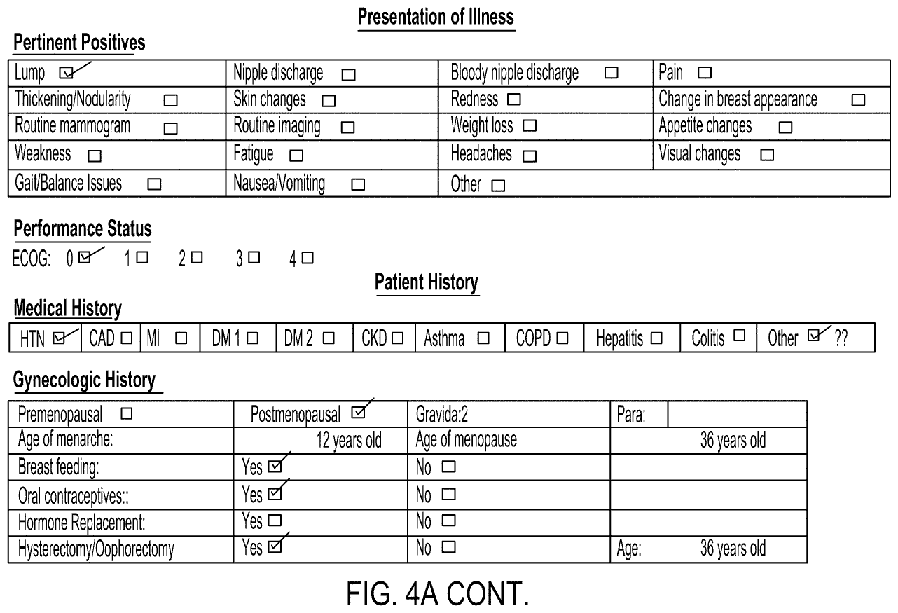

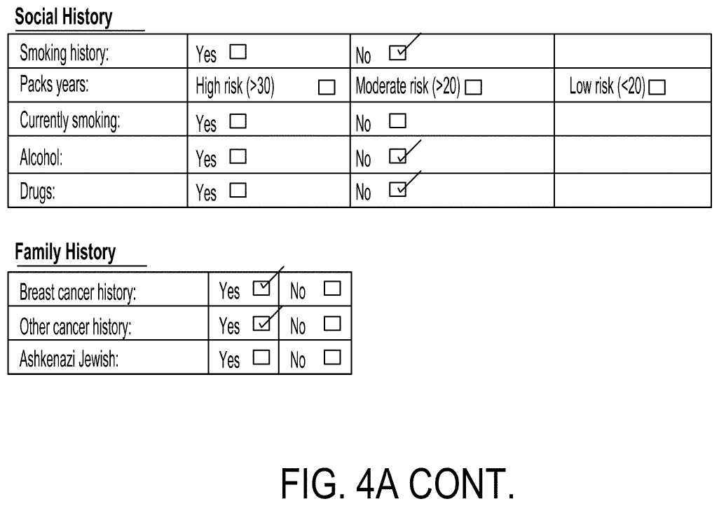

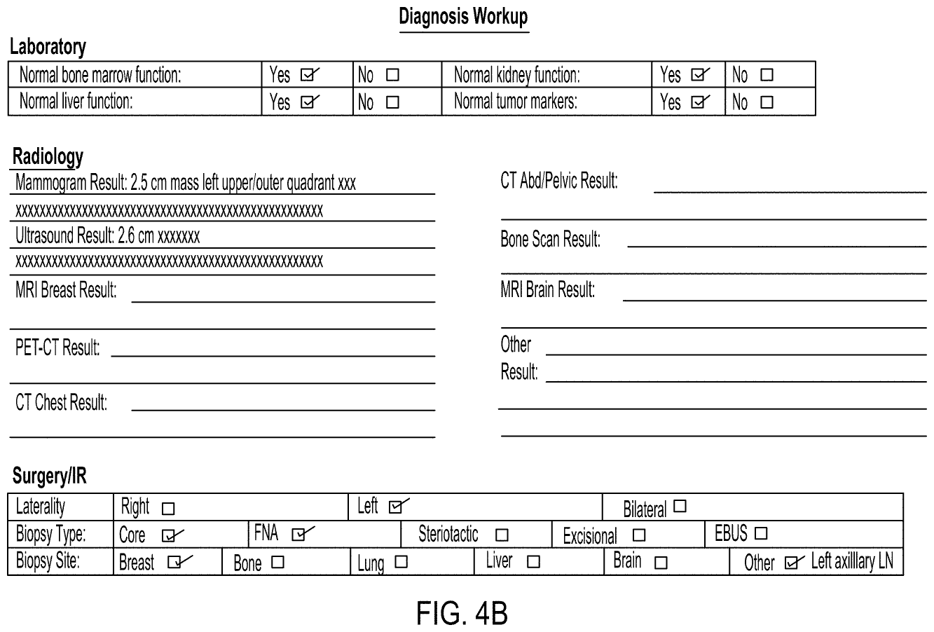

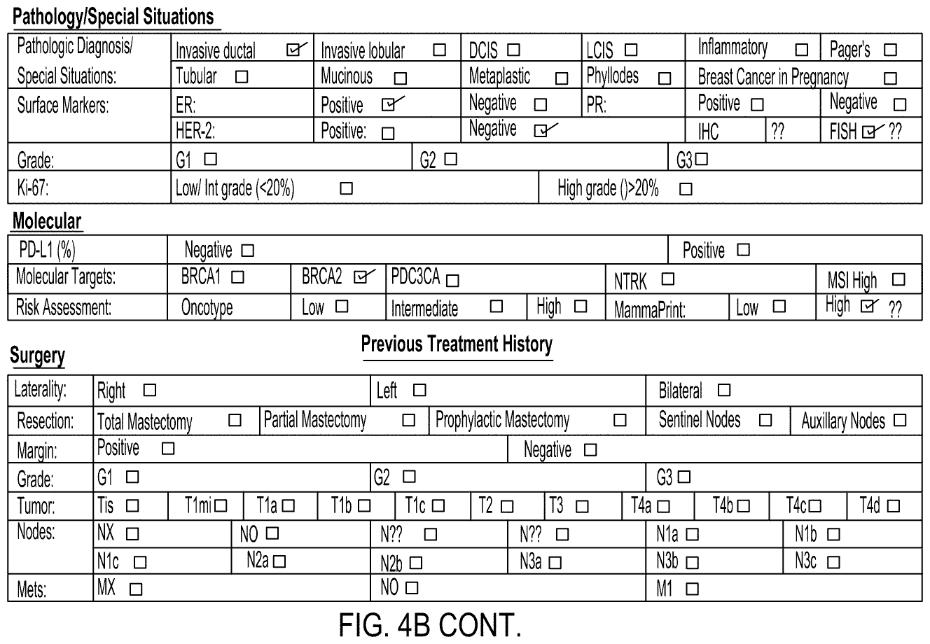

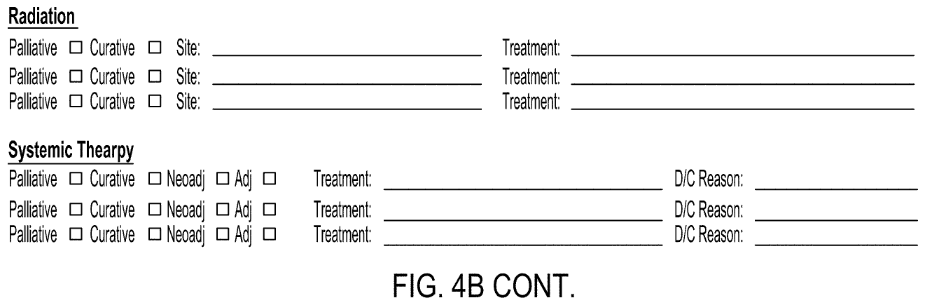

FIG. 2 illustrates a method 200 for expert group scenario selection and FIGS. 3 and 4 illustrate more details of the processes performed by the expert group scenario selection system 100 and in particular the backend 104. In the method 200, the scenario submission and capture for each user is performed (202). In one embodiment as shown in FIG. 3, the system 100 in FIG. 2 may implement this process using the front end 102 and in particular the case submission client on the computing device 102A and the web server 104A of the backend 104. As shown in FIG. 3, the submission process 202 may include the submission of a scenario intake form and other files related to the submitted scenario for consideration by an expert panel. The other files may be one or more images, PDF files, Word documents, spreadsheets and the like, and include any digital file that provides further details of the scenario being submitted. An example of the intake form for an embodiment of the system in which the expert panel is a medical oncology panel are shown in FIGS. 4A and 4B. In some embodiments like the example in FIGS. 4A and 4B, the intake form may have a number of checkboxes that makes it easier to interpret the filled-out form using a computer and for the person submitting the intake form (a patient, a provider for the patient or other party in the medical expert board example) to complete the fields needed by the system.

Once the scenario submission is completed, the method then can perform scenario processing (204). This process 204, like the submission process 202, is performed for each scenario that is being submitted. In one embodiment shown in FIG. 3, the scenario processing 204 may be performed by elements of the backend 104 and may perform two subprocesses. The first subprocess may route the “other files” to an optical character recognition (OCR) and analysis process 302. This process 302 may be implemented by a plurality of lines of computer code/instructions executed by a processor of a computer of the back end 104 to configure the processor to: 1) perform a text extraction process (such as OCR in one embodiment, on any of the files that are susceptible to having text extracted from them (an example being a PDF file); and 2) performing an analysis of the extracted text. The extracted text may be stored in the storage 104C along with the other data for the particular scenario.

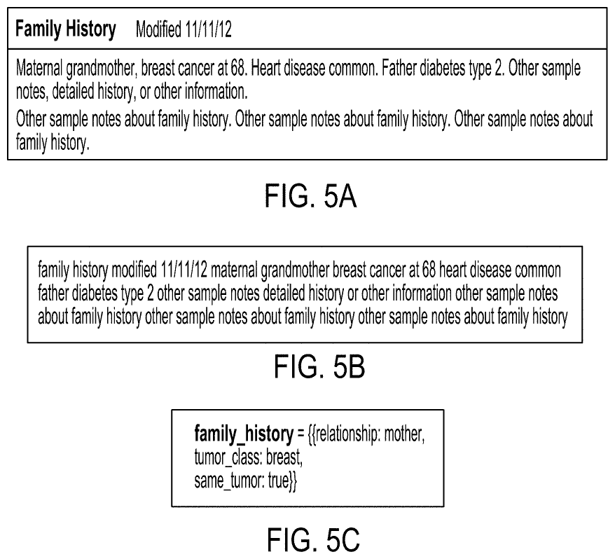

The second subprocess, like the above OCR process, is implemented by a plurality of lines of computer code/instructions executed by a processor on a computer of the back end 104 to configure the processor to perform a process. Using the business logic 104B of the backend 104, the second subprocess performs a feature extraction process in which one or more features of interest are extracted from the scenario data. The one or more extracted features of interest are stored in the storage 104C with the other scenario data. An example of this feature extraction subprocess for medical oncology data scenario submissions is shown in FIGS. 5A-5C. FIG. 5A shows the raw data in the scenario data while FIG. 5B shows the OCRed data extracted from the raw scenario data. FIG. 5C shows an example of a feature (family history which is an important feature of interest in medical oncology) that is extracted from the scenario data and stored in the storage.

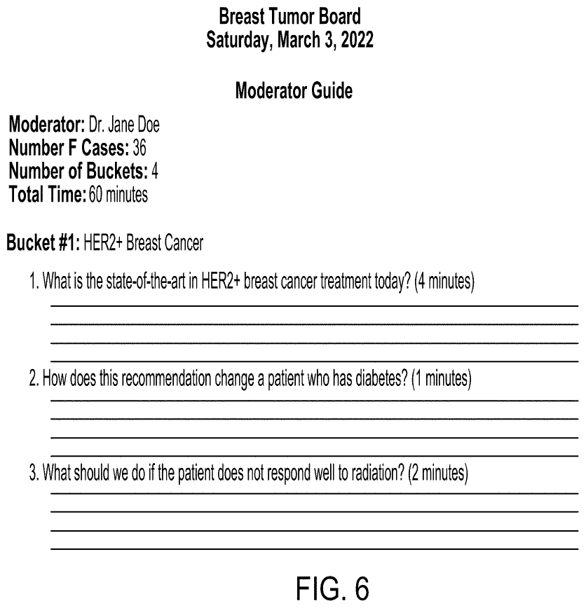

Returning to FIG. 2, the method may generate a scenario(s) docket/agenda for each expert board (206) in which the scenario(s) for a particular bucket are selected and gathered using all the scenarios placed into the particular bucket when this process is being performed. FIG. 6 shows an example of an agenda/docket that may be generated for medical oncology scenarios. In the example in FIG. 6, the expert is Dr. Jane Doe, the number of scenarios (known as cases for the medical oncology example use), the number of buckets being reviewed, and the time allotted are shown. Then, for each bucket (such as the HER2+ breast cancer bucket as shown in FIG. 6), the method generates one or more discussion topics based on the processes described above and a discussion time to ensure that the expert/panel of experts can complete all of the scenarios. The time allotted to each question for each bucket is calculated based on the topic being discussed. In the medical oncology use example in FIG. 6, a “state of the art treatment” question is allotted 4 minutes (quite of bit of information to discuss), but 1 minute is allotted for the topic of a treatment recommendation for a patient with diabetes since the recommendation is straightforward and affects fewer patients in the bucket. Thus, each topic and its allotted time is generated through a docket generation process 304 in the back end 104 that may again be implemented by a plurality of lines of computer code/instructions executed by a processor of a computer of the back end 104 to configure the processor to perform a process.

In some instances, the above method can scale to handle a large number of scenarios in each bucket without having to change/adjust the above process. For illustration, an example of the scalability for oncology is now provided, but this same scalability would exist for all other use cases. In traditional tumor boards, the session lasts approximately 1 hour and anywhere from 6-10 cases may be discussed. In many of the previous solutions discussed above, providers may discuss their cases with their second opinion provider for anywhere from 10 to 30 minutes. In general, various methods can allow for a discussion of anywhere between 2-10 cases per hour, and there is no clear opportunity for scaling. Assuming the best possible case scenario of 10 cases per hour, which is an average of 6 minutes per case. For 30 cases, discussions can last 180 minutes or 3 hours.

In the above disclosed method, if there is a bucket with 30 cases in it, the first 2 minutes of the round table discussion can be dedicated to discussing high-level treatment recommendations that are applicable to all 30 cases. Now, let's assume that in this bucket, there are four additional binary contextual variables that would affect the recommended treatment protocol (e.g., the presence of comorbidity X, whether the stage is higher or lower than 3, the presence of a specific mutation, etc.) These variables would have been identified by a similarity-difference process' analysis of the contextual information stored about each case in the bucket. There can be 2¹′4=16 possible combinations of these binary variables, so at most 16 possible aberrations from the general recommended treatment need to be discussed. Assuming that it takes about 1 minute to discuss one of these 16 possibilities and we have to discuss all of them, all 30 possible scenarios can be discussed in 2+16=18 minutes. This is a 10× speed-up over the traditional approach—an entire order of magnitude. Whether these possibilities are discussed in a branching, tree-like fashion or each of the 16 possibilities is just discussed sequentially won't affect the length of the discussion; it will still be 2+16 minutes. Often, many of these 2\hat{ }4 combinations of binary variables won't be represented by any of the scenarios in the bucket, further decreasing the time taken to discuss all 30 cases. Thus, the disclosed method provides scalability and can perform the task more rapidly than many processes due to the technical processes described above that allow the backend 104 to achieve these benefits.

The similarity-difference process may receive as input the known variables in an area of expertise and those known variables may affect a recommendation/outcome of the expert panel. The similarity-difference process may identify scenarios that match the known variables and scenarios that do not match the known variables. In the medical oncology example, the similarity-difference process may take the known variables that may affect oncology treatment recommendations and scan the cases in a particular bucket to see which cases match (on those variables) and which ones are different. In a simple example, if the only two important variables are diabetes and allergies and if there are 4 cases in the bucket, the similarity-difference process determines how many cases exist of [non-diabetic, non-allergic], [diabetic, non-allergic], [non-diabetic, allergic], [diabetic, allergic] since these will be the four branches/topics that need to be discussed by the expert panel. However, it is possible that there are only cases in the [non-diabetic, allergic] and [non-diabetic, non-allergic] groups for a particular bucket and then the agenda generated for the particular bucket (and used by the moderator) will not ask about diabetes that simplifies the discussion by the expert panel. In many systems, the diabetes topic would first be discussed and then the expert panel would realize that there are no cases being discussed that have the diabetes variable.

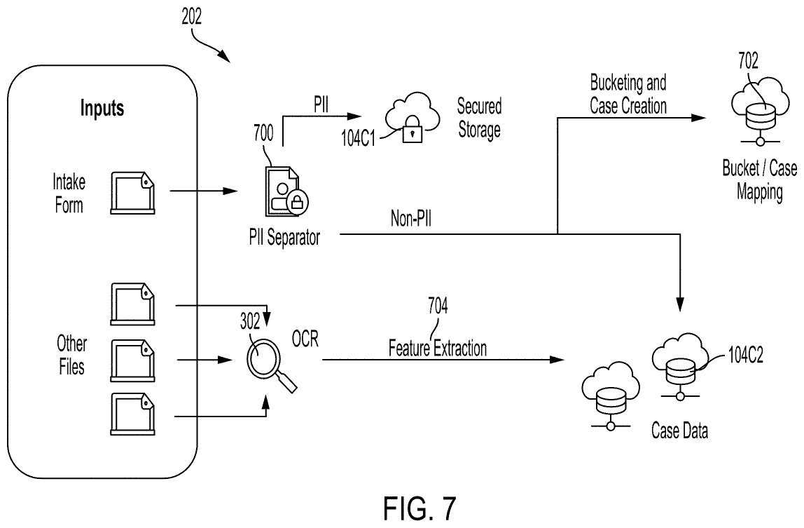

FIG. 7 illustrates further details of the scenario submission process 202 and the cloud storage 104C of the system. As discussed above, the inputs to this process are the scenario intake form and the unstructured one or more other files submitted with the scenario. There are three main components of the cloud database case storage system 104C that allow the system to work efficiently.

First, as shown in FIG. 7, the system may have a personal identifiable information (PII) separator process 700 that may be implemented as a plurality of lines of computer code/instructions executed by a processor of a computer system of the storage system 104C. When a scenario is submitted, a unique case ID is created and serves as the primary key across most tables. The PII separator process separates any/all of the private/PII information from the intake form from the remaining features and stores that PII information a secured storage 104C1 in separate tables with separate access requirements. This process is necessary for use cases in which the expert panel is discussing scenarios that involve PII information, such as a tumor board. The non-PII information extracted from the intake form may be stored in a normal storage system 104C2 as case data.

Second, the non-PII information for each scenario may be used by a bucketing and case creation process 702. This process also may be implemented as a plurality of lines of computer code/instructions executed by a processor of a computer system of the back end 104 that configures the computer system or processor to perform the bucketing process. This process may also be called bucket tagging in which each scenario may be assigned a bucket, based on the information provided in the intake form, and thus all case data rows are tagged with a bucket ID to allow for fast generation of roundtable dockets as discussed below. Note that for each different use case, a different bucket schema will be needed. For example, in the oncology use case, the buckets differentiate different types of tumors as well as unique characteristics of each type of tumor. In a use case for a different area of expertise, the bucket schema may be different.

Third, the system and process 202 may perform an automatic feature extraction process 704 from the unstructured one or more files submitted with the scenario. This process also may be implemented as a plurality of lines of computer code/instructions executed by a processor of a computer system of the back end 104 that configures the computer system or processor to perform the feature extraction process. For each of the one or more file(s) submitted for a scenario, the system may store the raw file (with the necessary security levels) as well as an OCR-generated (process 302) version of the contents.

To take one example use case, 50 additional features may be identified that are potentially relevant to a discussion for an area of expertise but are not included in the intake form and, for each feature, this process may perform one of multiple text analytics/natural language processing (NLP) techniques to attempt to extract that feature from unstructured text wherein each feature is particular data that may be extracted from each scenario. An example of a feature that may be extracted using the above techniques in the medical area of expertise may be whether or not the patient was hospitalized. This feature is not explicitly on the intake form, but the system can analyze the one or more additional files submitted using the above techniques to find words/phrases/other indications that the patient was hospitalized. The system can then add another column/feature (hospitalized=true or false in this example) to the data. The one or more feature(s) discovered using the above techniques may be applicable to a single bucket of multiple buckets in the area of expertise. As will be apparent, the features for each different area of expertise may be very different so that hospitalization would not be a feature for the finance or architecture area of expertise. For example, in the architecture example, a feature may be the presence of a heat pump in a residence that again may not be explicitly in the intake form but can be discovered by the system.

The process 704 may use one or more simple regex keyword searches, complex language models based on deep neural networks, Bayesian topic models, FastText, lexical network analysis, Word2Vec, GloVe, etc. that together include neural networks, topic models, Bayesian models, graphical models, and other vector-space models. The one or more text analytics methods selected may be custom selected for each feature. In one embodiment, the text analytics method for each feature may be manually created to best capture that feature from the unstructured data of the one or more additional files. Then, for each submitted scenario, the process will initialize a feature vector (of length 50, in this example) of all NULL values. Next, we will loop over each submitted file, apply OCR to it, and then run each feature extraction algorithm over the text, and if we are able to extract information related to that feature, we will update the feature vector in the corresponding index. Otherwise, we will leave it NULL. We will end up with feature vector for each case that is a mixture of NULL values (features that were not detected) and feature values of various data types (e.g., dictionaries for family histories, Boolean values for the presence of comorbidities, etc.) These vectors will then be combined with the contextual data provided in the intake form to create entries in the Case Data tables.

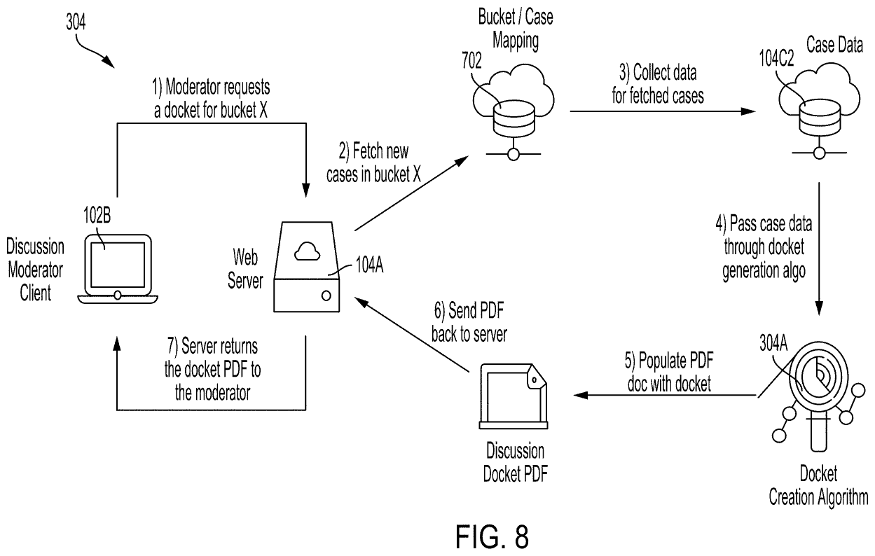

FIG. 8 illustrates further details of the scenario docket generation and expert panel discussion processes 304. During the process 304, a moderator (using a moderator client on a computing device 102B) may request a docket/agenda for a particular bucket X. The web server 104A of the backend 104 may fetch all of the scenarios currently in bucket X and the bucket/case mapping 702 may collect the data for those fetched scenarios (known as cases in the medical expert panel example) from the storage 104C2 that is then passed onto a docket generation process 304A. This process 304A may be implemented as a plurality of lines of computer code/instructions executed by a processor of a computer system of the back end 104 that configures the computer system or processor to perform the docket generation process as described below. As shown in FIG. 8, the results of the docket generation process may be used to populate a document, such as a PDF, that becomes a discussion docket for bucket X. The discussion docket for bucket X is sent to the web server 104A that communicates it to the moderator client in the computing device 102B so that the moderator and the expert panel can perform their tasks based on the discussion docket (an example of part of which is shown in FIG. 6 and described above.

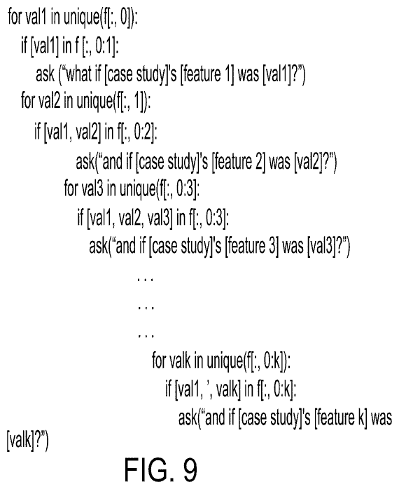

FIG. 9 shows the pseudocode of a portion of the docket generation process of the topics for the scenario docket/agenda. During the docket generation process 304A, each scenario/case ci in bucket B (with n cases, 1<=i<=n) has a corresponding feature vector vi comprised of all of the features that the system can extract from the intake form and the associated unstructured uploads as described above. After collecting a baseline of ˜100 cases (not necessarily in one bucket) and putting them through expert panel reviews, the system is able to create feature vectors wi for each case i called “expert recommendation vectors.” For example, in the oncology use case, this may correspond to a boolean vector of length 3 with features [‘radiation’, ‘chemo’, ‘surgery’]=[true, true, false]. By training a random forest model to predict wi from vi for all i, the method can calculate the relative importance of the features of v in predicting expert recommendations.

To generate a docket, the process may begin by asking a general question soliciting recommendations for any case in bucket B (see HER2 overview and treatment question in FIG. 6). It should be remembered that all buckets are designed such that the cases in the same bucket will share some key similarities. The method may set a threshold k of the number of variables. The threshold is selected based on a complexity/length of the expert panel discussion for each bucket in each area of expertise and may range between 3 to 8. More likely, k will range from 4 to 5 in most instances.

Using the threshold k, the method may then select the k features with the highest variable importance scores from the random forest model to construct a subvector fi for each case ci. The jth element of fi is the ith case's value for the jth most important feature from the random forest. Let f be the matrix with row i of f=fi. The method, based on the above, then generates topics for the features in all of the cases in the bucket (again see example in FIG. 6) and may recursively ask questions as shown in FIG. 9. The process shown in FIG. 9 may be termed a backtracking process that guarantees that every branch of questions results in at least one of the cases being fully addressed, but since not every feature will necessarily impact the treatment recommendation, the system will iterate over all of the relevant features/topics very quickly.

After the panel has completed its recommendations for the cases in the bucket, the method may codify the expert recommendations as new wi vectors and retrain the random forest to generate a fresh set of “most important variables”. This training loop will continually improve the ability of the process to only ask questions about relevant features. For example, in the oncology use case, the system performs an analysis by taking all of the features from all of the cases that the system has already done to build a model to predict the recommended treatment(s). These recommended treatments may be as simple as a three element boolean vector such [radiation=false, chemo=true, surgery=false]. In building this model, the system may discover that some features are more predictive than others (i.e., some features have a larger impact on the treatment regimen) and these features are the key variables. Moving forward, the docket creation process knows that these are the key variables to discuss when creating topic agendas. Then, the system hosts more tumor boards and collects more data. With this additional data, the analysis above is updated potentially discovering more or better key variables.

Method for Docket Generation

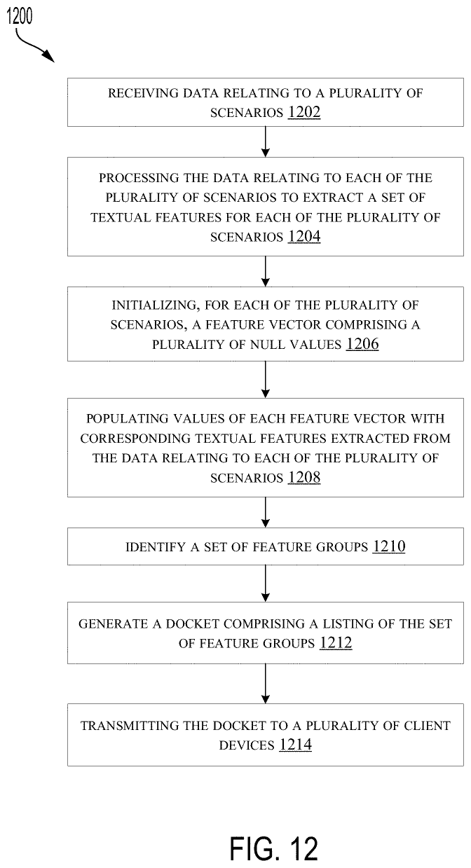

As described above, the present embodiments relate to generation of a docket of feature groups based on a plurality of identified features from a dataset. FIG. 12 illustrates an example method for generation of a docket. For instance, the method can be performed by a computing device (or series of interconnected computing devices) implementing a docket generation model as described herein.

At 1202, the method can include receiving data relating to a plurality of scenarios. Each scenario can include a portion of data, such as data relating to a patient or a ticket generated for a datacenter, for example. Each of the scenarios can include one or more corresponding supporting files. Example supporting files can include medical records, metadata relating to a ticket, graph or chart data, etc. In some instances, the scenarios relate to medical cases, and the supporting files comprise medical documents. In other instances, the scenarios comprise automatically generated tickets relating to a datacenter, and the supporting files comprise data relating to the automatically-generated ticket.

At 1204, the method can include processing the data relating to each of the plurality of scenarios to extract a set of textual features for each of the plurality of scenarios. Each extracted textual feature can include a feature specific to a scenario. An example textual feature can include a condition (e.g., a medical condition) or measured value (e.g., a test result) relating to a first scenario (e.g., a patient). Another example textual feature can relate to an application or network device within a datacenter that corresponds to a given ticket.

In some instances, processing the data relating to each of the plurality of scenarios to extract the set of textual features for each of the plurality of scenarios further comprises converting each supporting file into unstructured text. The processing can be performed on the unstructured text of each supporting file. Further, processing the data relating to each of the scenarios on the unstructured text of each supporting file further can include using a text analytics process to programmatically extract each textual feature from the unstructured text of each supporting file.

At 1206, the method can include initializing, for each of the plurality of scenarios, a feature vector comprising a plurality of null values. The null values can be replaced with values as relevant textual features are identified.

At 1208, the method can include populating values of each feature vector with corresponding textual features extracted from the data relating to each of the plurality of scenarios. In some instances, a feature vector can include both null values and populated values. The portions of the feature vector comprising null values can include textual feature types not identified for a specific scenario.

In some instances, each textual feature is populated in each corresponding feature vector according to a location of the textual feature identified from each corresponding supporting file. For example, a mapping can be generated specifying a feature type assigned to each value in the feature vector. As an example, a first value can be assigned a device ID specifying a device in a datacenter relating to a ticket.

At 1210, the method can include processing each of the feature vectors to identify a set of feature groups. A docket generation model can process the feature vectors as described herein. Each feature group can include a set of scenarios that include one or more extracted textual features that are common across feature vectors corresponding to each scenario of the set of scenarios. A first example feature group can include a group of scenarios comprising a common medical condition across the group of scenarios. Another example feature group can include a group of tickets comprising a commonly identified network device that originated a ticket in a datacenter. The feature groups can be identified based on comparing feature vector values in the set of feature vectors for each scenario.

At 1212, the docket generation model can generate a docket comprising a listing of the set of feature groups. The listing of the set of feature groups can be arranged by a number of scenarios corresponding to each of the feature groups and/or a number of extracted textual features for each of the set of feature groups.

In some instances, processing each textual feature part of each of the feature groups can use a feature hierarchy to generate a cumulative feature score of each feature group. A feature hierarchy can include a multi-level hierarchy of features, with each feature assigned weights and/or values for use in identifying a priority of each feature group. In some instances, the feature hierarchy can utilize a tree structure, and the arrangement of feature groups on the docket can be based on a location of each feature group in the tree structure. The arrangement of the feature groups can be based at least on the cumulative feature score of each feature group. In some instances, the docket generation model includes a random forest model. In some instances, the listing of feature groups are presented as sentences that include the features common to the feature groups.

At 1214, the method can include transmitting the docket to a plurality of client devices. In some instances, the method can include obtaining a response for each of the set of feature groups listed in the docket from any of the client devices.

In some instances, the method can include training the docket generation model using the feature vectors, the set of feature groups, and the responses for each of the set of feature groups to generate a feature hierarchy of the extracted textual features in the feature vectors.

In another example embodiment, a system is provided. The system can include a processor and a computer-readable medium. The computer-readable medium can comprise instructions that, when executed by the processor, cause the processor to receive data relating to a plurality of scenarios. Each of the scenarios can comprise one or more corresponding supporting files.

The instructions can further cause the processor to process the data relating to each of the plurality of scenarios to extract a set of textual features for each of the plurality of scenarios. Processing the data further can include converting each supporting file into unstructured text.

The instructions can further cause the processor to populate, for each of the plurality of scenarios, values of a corresponding feature vector with corresponding textual features extracted from the data relating to each of the plurality of scenarios. Each textual feature can be populated in each corresponding feature vector according to a location of the textual feature identified from each corresponding supporting file.

The instructions can further cause the processor to process each of the feature vectors to identify a set of feature groups, each feature group comprising a set of scenarios that include one or more extracted textual features that are common across feature vectors corresponding to each scenario of the set of scenarios. The instructions can further cause the processor to generate a docket comprising a listing of the set of feature groups. The listing can be arranged by a number of scenarios corresponding to each of the feature groups and/or a number of extracted textual features for each of the set of feature groups.

The instructions can further cause the processor to transmit the docket to a plurality of client devices.

In some instances, processing the data relating to each of the scenarios on the unstructured text of each supporting file further comprises using a text analytics process to programmatically extract each textual feature from the unstructured text of each supporting file.

In some instances, the instructions further cause the processor to process each textual feature part of each of the feature groups using a feature hierarchy to generate a cumulative feature score of each feature group, wherein the arrangement of the feature groups is based at least on the cumulative feature score of each feature group.

In some instances, the instructions further cause the processor to obtain a response for each of the set of feature groups listed in the docket from any of the client devices.

In some instances, the instructions further cause the processor to train a docket generation model using the feature vectors, the set of feature groups, and the responses for each of the set of feature groups to generate a feature hierarchy of the extracted textual features in the feature vectors.

In another example embodiment, a computer-implemented method is provided. The computer-implemented method can include receiving at data relating to a plurality of scenarios. Each of the scenarios can comprise one or more corresponding supporting files.

The computer-implemented method can also include processing the data relating to each of the plurality of scenarios to extract a set of textual features for each of the plurality of scenarios.

The computer-implemented method can also include populating, for each of the plurality of scenarios, values of a corresponding feature vector with corresponding textual features extracted from the data relating to each of the plurality of scenarios.

The computer-implemented method can also include processing, by a docket generation model, each of the feature vectors to identify a set of feature groups, each feature group comprising a set of scenarios that include one or more extracted textual features that are common across feature vectors corresponding to each scenario of the set of scenarios and generate a docket comprising a listing of the set of feature groups. The listing can be arranged by a number of scenarios corresponding to each of the feature groups and/or a number of extracted textual features for each of the set of feature groups.

The computer-implemented method can also include transmitting the docket to a plurality of client devices.

The computer-implemented method can also include obtaining a response for each of the set of feature groups listed in the docket from any of the client devices. The computer-implemented method can also include training the docket generation model using the feature vectors, the set of feature groups, and the responses for each of the set of feature groups to generate a feature hierarchy of the extracted textual features in the feature vectors. The feature hierarchy can be used in arranging the listing of the set of feature groups.

In some instances, processing the data relating to each of the plurality of scenarios to extract the set of textual features for each of the plurality of scenarios further comprises converting each supporting file into unstructured text. The processing can be performed on the unstructured text of each supporting file.

In some instances, processing the data relating to each of the scenarios on the unstructured text of each supporting file further comprises using a text analytics process to programmatically extract each textual feature from the unstructured text of each supporting file.

In some instances, each textual feature is populated in each corresponding feature vector according to a location of the textual feature identified from each corresponding supporting file.

### Tumor Board Use Case

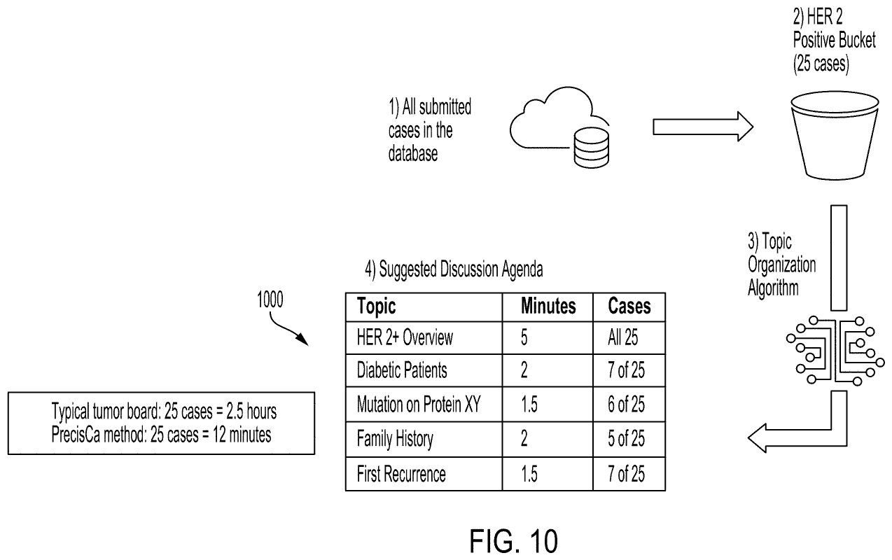

In a use case in which the above described system and method is being used for a tumor board that is discussing oncology treatment recommendations for different tumors, the PII separation process is very important at the intake stage. Oncology providers who submit the different scenarios for the tumor of each patient will use the online intake form to answer questions about complex tumor cases, attaching additional Electronic Medical Records (EMRs). The intake process carefully separates case data from PII and store the values accordingly as discussed above. In this use case, each bucket may correspond to different tumor subtypes within individual sites (e.g., Stage II Breast, etc.). The review panel will consist of one or more experts who are considered leaders in the field of oncology research and hail from prestigious institutions. FIG. 10 illustrates an example of the method when applied to the medical oncology use case. In the example in FIG. 10, the particular bucket for discussion is a bucket containing scenarios with breast tumors that are HERs positive and includes 25 cases. Based on this bucket, the system generates the docket/agenda/topics 1000 for this bucket. As shown in FIG. 10, the agenda may include both topics, the time allocated for each topic and the number of cases to which each topic is relevant. Also note that the topics generated by the system are specific to the area of expertise (HER2 positive breast cancer tumors) and thus include, in the example in FIG. 10, an overview of HER2+, diabetic patients, mutation on protein X-Y, family history, and information on first recurrence. Because of the original bucketing and then the agenda generation in view of the buckets, a panel of experts can review and provide recommendations/second opinions for the 25 exemplary cases in 12 minutes as compared to another tumor board that can spend 2.5 hours discussed those same 25 exemplary cases.

The foregoing description, for purpose of explanation, has been with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include and/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond those set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or the modules can comprise programming instructions transmitted to a general-purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software, and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again this does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

What is claimed is:

1. A method comprising:
receiving, at a computer system, data relating to a plurality of scenarios, wherein each of the scenarios comprise one or more corresponding supporting files;
processing the data relating to each of the plurality of scenarios to extract a set of textual features for each of the plurality of scenarios;
initializing, for each of the plurality of scenarios, a feature vector comprising a plurality of null values;
populating values of each feature vector with corresponding textual features extracted from the data relating to each of the plurality of scenarios;
processing, by a docket generation model, each of the feature vectors to:
identify a set of feature groups, each feature group comprising a set of scenarios that include one or more extracted textual features that are common across feature vectors corresponding to each scenario of the set of scenarios; and
generate a docket comprising a listing of the set of feature groups, the listing arranged by a number of scenarios corresponding to each of the feature groups and/or a number of extracted textual features for each of the set of feature groups; and
transmitting the docket to a plurality of client devices.

2. The method of claim 1, wherein processing the data relating to each of the plurality of scenarios to extract the set of textual features for each of the plurality of scenarios further comprises converting each supporting file into unstructured text, wherein the processing is performed on the unstructured text of each supporting file.

3. The method of claim 2, wherein processing the data relating to each of the scenarios on the unstructured text of each supporting file further comprises using a text analytics process to programmatically extract each textual feature from the unstructured text of each supporting file.

4. The method of claim 1, wherein each textual feature is populated in each corresponding feature vector according to a location of the textual feature identified from each corresponding supporting file.

5. The method of claim 1, further comprising:
obtaining a response for each of the set of feature groups listed in the docket from any of the client devices.

6. The method of claim 5, further comprising:
training the docket generation model using the feature vectors, the set of feature groups, and the responses for each of the set of feature groups to generate a feature hierarchy of the extracted textual features in the feature vectors.

7. The method of claim 6, further comprising:
processing each textual feature part of each of the feature groups using the feature hierarchy to generate a cumulative feature score of each feature group, wherein the arrangement of the feature groups is based at least on the cumulative feature score of each feature group.

8. The method of claim 1, wherein the docket generation model includes a random forest model.

9. The method of claim 1, wherein the scenarios relate to medical cases, and the supporting files comprise medical documents.

10. The method of claim 1, wherein the scenarios comprise automatically-generated tickets relating to a datacenter, and wherein the supporting files comprise data relating to the automatically-generated ticket.

11. The method of claim 1, wherein the listing of feature groups are presented as sentences that include the features common to the feature groups.

12. A system comprising:
a processor; and
a computer-readable medium comprising instructions that, when executed by the processor, cause the processor to:
receive data relating to a plurality of scenarios, wherein each of the scenarios comprise one or more corresponding supporting files;
process the data relating to each of the plurality of scenarios to extract a set of textual features for each of the plurality of scenarios, wherein processing the data further comprises converting each supporting file into unstructured text;
populate, for each of the plurality of scenarios, values of a corresponding feature vector with corresponding textual features extracted from the data relating to each of the plurality of scenarios, wherein each textual feature is populated in each corresponding feature vector according to a location of the textual feature identified from each corresponding supporting file;
process each of the feature vectors to:
identify a set of feature groups, each feature group comprising a set of scenarios that include one or more extracted textual features that are common across feature vectors corresponding to each scenario of the set of scenarios; and
generate a docket comprising a listing of the set of feature groups, the listing arranged by a number of scenarios corresponding to each of the feature groups and/or a number of extracted textual features for each of the set of feature groups; and
transmit the docket to a plurality of client devices.

13. The system of claim 12, wherein processing the data relating to each of the scenarios on the unstructured text of each supporting file further comprises using a text analytics process to programmatically extract each textual feature from the unstructured text of each supporting file.

14. The system of claim 12, wherein the instructions further cause the processor to:
process each textual feature part of each of the feature groups using a feature hierarchy to generate a cumulative feature score of each feature group, wherein the arrangement of the feature groups is based at least on the cumulative feature score of each feature group.

15. The system of claim 12, wherein the instructions further cause the processor to:
obtain a response for each of the set of feature groups listed in the docket from any of the client devices.

16. The system of claim 15, wherein the instructions further cause the processor to:
train a docket generation model using the feature vectors, the set of feature groups, and the responses for each of the set of feature groups to generate a feature hierarchy of the extracted textual features in the feature vectors.

17. A computer-implemented method comprising:
receiving at data relating to a plurality of scenarios, wherein each of the scenarios comprise one or more corresponding supporting files;
processing the data relating to each of the plurality of scenarios to extract a set of textual features for each of the plurality of scenarios;
populating, for each of the plurality of scenarios, values of a corresponding feature vector with corresponding textual features extracted from the data relating to each of the plurality of scenarios;
processing, by a docket generation model, each of the feature vectors to:
identify a set of feature groups, each feature group comprising a set of scenarios that include one or more extracted textual features that are common across feature vectors corresponding to each scenario of the set of scenarios; and
generate a docket comprising a listing of the set of feature groups, the listing arranged by a number of scenarios corresponding to each of the feature groups and/or a number of extracted textual features for each of the set of feature groups;
transmitting the docket to a plurality of client devices;
obtaining a response for each of the set of feature groups listed in the docket from any of the client devices; and
training the docket generation model using the feature vectors, the set of feature groups, and the responses for each of the set of feature groups to generate a feature hierarchy of the extracted textual features in the feature vectors, wherein the feature hierarchy is configured to be used in generating the arrangement of the listing of the set of feature groups of the docket.

18. The computer-implemented of claim 17, wherein processing the data relating to each of the plurality of scenarios to extract the set of textual features for each of the plurality of scenarios further comprises converting each supporting file into unstructured text, wherein the processing is performed on the unstructured text of each supporting file.

19. The computer-implemented of claim 18, wherein processing the data relating to each of the scenarios on the unstructured text of each supporting file further comprises using a text analytics process to programmatically extract each textual feature from the unstructured text of each supporting file.

20. The computer-implemented method of claim 17, wherein each textual feature is populated in each corresponding feature vector according to a location of the textual feature identified from each corresponding supporting file.
